(19) United States
(12) Patent Application Publication
(10) Pub. No.: US 2024/0279682 A1
(43) Pub. Date: Aug. 22, 2024

(54) AAV Manufacturing Methods

(71) Applicant: MeiraGTx UK II Limited, London (GB)

(72) Inventors: Florian Dziopa, London (GB); Ana Valinhas, London (GB); Bastiaan Leewis, London (GB)

(73) Assignee: MeiraGTx UK II Limited, London (GB)

(21) Appl. No.: 18/571,478

(22) PCT Filed: Jun. 17, 2022
(86) PCT No.: PCT/IB2022/000347

§ 371 Date: Dec. 18, 2023

Related U.S. Application Data
(60) Provisional application No. 63/211,877, filed on Jun. 17, 2021.

(51) Int. Cl. C12N 15/86

(52) U.S. Cl. CPC C12N 15/86; C12N 2750/14143; C12N 2750/14152

(57) ABSTRACT

Methods for producing recombinant adeno-associated virus (rAAV) particles include: an expansion phase including increasing the number of cells in at least one culture vessel containing culture medium; introducing into the cells a first polynucleotide sequence including a transgene flanked by AAV inverted terminal repeats, and optionally a second polynucleotide sequence including AAV rep and cap genes, and/or a third polynucleotide sequence including one or more helper genes; a production phase including culturing the cells into which the one or more polynucleotide sequences were introduced and adding calcium (Ca) ions (i.e., salts of Ca2+) to the production phase culture medium; and isolating the rAAV particles.

### CROSS-REFERENCE TO RELATED APPLICATIONS

This application claims the benefit of priority to U.S. Provisional Patent Application No. 63/211,877, filed Jun. 17, 2021, which is incorporated herein by reference in its entirety.

### FIELD

The present disclosure provides methods for making recombinant adeno associated virus (AAV) vectors by adding certain salts, in particular calcium salts, e.g., calcium chloride, to the AAV production medium.

### BACKGROUND

Adeno-associated virus (AAV) is a replication-deficient parvovirus. AAV particles comprise a capsid having three capsid proteins—VP1, VP2 and VP3—enclosing a single-stranded DNA genome of about 4.8 kb in length, which may be either the plus or minus strand. Particles containing either strand are infectious, and replication occurs by conversion of the parental infecting single strand to a duplex form, and subsequent amplification, from which progeny single strands are displaced and packaged into capsids.

AAV is dependent on co-infection with other viruses, mainly adenoviruses, in order to replicate. Its single-stranded genome contains three genes, rep (Replication), cap (Capsid), and aap (Assembly), which give rise to at least nine gene products through the use of three promoters, alternative translation start sites, and differential splicing. These coding sequences are flanked by inverted terminal repeats (ITRs) that are required for genome replication and packaging. The rep gene encodes four proteins (Rep78, Rep68, Rep52, and Rep40), which are involved in viral genome replication and packaging, while cap expression gives rise to the viral capsid proteins (VP1, VP2, and VP3), which form the outer capsid shell that protects the viral genome, as well as being actively involved in cell binding and internalization. The aap gene encodes the assembly-activating protein (AAP) in an alternate reading frame that overlaps the cap gene. This AAP protein is thought to provide a scaffolding function for capsid assembly.

AAV particles have features that make them attractive as vectors for therapeutic applications including gene therapy and genetics vaccines. AAV infects a wide range of cell types including many mammalian cells, allowing the possibility of targeting many different tissues in vivo. AAV infects slowly dividing and non-dividing cells. For therapeutic applications, recombinant AAV (rAAV) are used in which the genome includes a heterologous transgene and typically retains the ITRs, but lacks the viral rep, cap, and aap genes. In the absence of Rep proteins, ITR-flanked transgenes can form transcriptionally active nuclear extrachromosomal element or episome that can persist essentially for the lifetime of the transduced cells.

Cell-culture systems for production of rAAV vectors include transient transfection of human cell lines and infection of mammalian or insect cell lines. rAAV vector generation methods generally include a producer cell type that provides the biosynthetic machinery for vector generation, combined with helper vectors (for example, helper plasmids, helper viruses) as the source for additional gene products required for rAAV replication and packaging.

Important goals for the rAAV vector production method are to achieve consistent, high vector productivity while minimizing generation of product-related impurities, including AAV-encapsidated residual DNA impurities and empty capsids. Measured as vector genomes (VG) generated per cell, rAAV vector productivity can be highly variable, ranging from less than 10³ to 2×10⁵ VG per cell. In addition to higher cost-effectiveness, an important advantage of high productivity is that purification can be more efficient when the starting material has a higher ratio of the rAAV vector product to total harvest biomass.

Product-related impurities resemble the rAAV vector itself and cannot easily be separated from rAAV vectors during the purification process. AAV empty capsids are generated at high levels in current rAAV vector production systems. For AAV2, 50 to 95% of total AAV particles generated in cell culture are empty capsids. However, to limit potential deleterious immune responses to AAV capsids it is prudent to minimize the generation of empty capsids in cell culture and/or substantially remove them during vector purification. Accordingly, there is a need for methods that increase rAAV productivity and/or decrease product related impurities such as empty capsids.

### SUMMARY

The present invention provides methods for producing recombinant adeno associated virus (rAAV) particles by adding certain salts, in particular calcium salts, e.g., calcium chloride, to the AAV production medium. Described herein is a method for producing recombinant adeno-associated virus (rAAV) particles, wherein the method includes:
(i) an expansion phase comprising increasing the number of cells in at least one culture vessel containing culture medium;
(ii) introducing into the cells a first polynucleotide sequence including a transgene flanked by AAV inverted terminal repeats (ITRs), and optionally a second polynucleotide sequence including AAV rep and cap genes, and/or a third polynucleotide sequence including one or more helper genes;
(iii) a production phase (in which rAAV particles are produced) comprising culturing the cells from step (ii) and adding calcium (Ca) ions (i.e., salts of Ca²⁺) to the culture medium at about 0 to about 24 hours after introduction of the first polynucleotide sequence such that the total concentration of Ca ions in the culture medium is greater than 0.3 mM and less than 10 mM; and
(iv) isolating the rAAV particles.

In embodiments of the method, adding Ca ions to the culture medium includes adding a Ca salt such as CaCl₂). Calcium ions can be added to a concentration of Ca ions in the culture medium between about 0.5 mM to about 9 mM, between about 1 mM to about 9 mM, between about 1 mM to about 8 mM, between about 1 mM to about 7 mM, between about 2 mM to about 9 mM, between about 2 mM to about 8 mM, between about 2 mM to about 7 mM, between about 2 mM to about 6 mM, between about 2 mM to about 5 mM, or between about 2 mM to about 4 mM. In embodiments, calcium ions are added to a concentration of about 1 mM, about 1.5 mM, about 2 mM, about 2.5 mM, about 3 mM, about 3.5 mM, about 4 mM, about 4.5 mM, about 5 mM, about 5.5 mM, about 6 mM, about 6.5 mM, about 7 mM, about 7.5 mM, about 8 mM, about 8.5 mM, about 9, or about 9.5 mM. In embodiments, calcium ions are added to the production culture medium to a concentration of about 0.5 mM to about 6 mM; about 2 mM to about 6 mM, about 2 mM to about 4 mM, or about 1 mM to about 3 mM.

In some embodiments, the Ca ions (Ca salt, e.g., CaCl₂)) are added after introduction of at least one of the first, second and third polynucleotide sequences or at the beginning of the production phase (i.e., at about 0 hours post-introduction). In embodiments, the Ca ions are added anytime from about 0 hours to about 24 hours (e.g., 0, about 1, about 2, about 5, about 8, about 12, about 18, about 23, about 24, about 24.5 hours) after introduction of the first polynucleotide sequence or after beginning of the production phase. In embodiments, the Ca ions are added anytime from about 0 hours to about 18 hours after introduction of the first polynucleotide sequence or after beginning the production phase. In embodiments, the Ca ions are added anytime from about 6 hours to about 20 hours, about 6 hours to about 18 hours, or about 6 to about 12 hours after introduction of the first polynucleotide sequence or beginning of the production phase. In some embodiments, the Ca ions are added at about 12 hours after introduction of at least one of the first, second and third polynucleotide sequences (or beginning of the production phase) such that the total concentration of Ca in the culture medium is about 2 mM.

In embodiments of the method, the expansion phase culture medium and/or the production phase culture medium are serum-free. In embodiments, the expansion and/or the production phase comprises adding one or more of glutamine, a glutamine precursor or an amino acid dipeptide including glutamine to the expansion phase culture medium and/or to the production phase culture medium. The one or more of glutamine, glutamine precursor or an amino acid dipeptide including glutamine can be one or more of, e.g., L-alanyl-L-glutamine, L-glutamine, glutamate, glycyl-L-glutamine, glutamine protein hydrolysate, L-glutamic acid, and a glutamine dipeptide. A solution including at least one of glutamine, glutamine precursor or an amino acid dipeptide including glutamine can be added to the production phase culture medium at, e.g., one or more of about 6 hours, about 12 hours, about 24 hours, about 48 hours, and/or about 72 hours after introduction of at least one of the first, second and third polynucleotide sequences one or more of about 6 hours, about 12 hours, about 24 hours, about 48 hours, and/or about 72 hours after beginning the production phase.

In embodiments of the method, the expansion phase and/or the production phase comprises adding anti-clumping supplement to the expansion phase culture medium and/or to the production phase culture medium. In embodiments, the anticlumping supplement comprises dextran sulfate, heparin and/or other sulfated glycosaminoglycans that suppress the aggregation of the cells. In embodiments, the anticlumping supplement comprises sodium heparin, which can be added to the media to concentrations of about 25 μg/ml to about 250 μg/ml, for example, about 25 μg/ml, about 50 μg/ml, about 100 μg/ml, about 150 μg/ml, and/or about 200 μg/ml. In embodiments, anticlumping supplement is not added to the production phase, or is only added at the end of the production phase shortly prior to the isolation step, e.g., within about 10 hours, within about 5 hours, within about 4 hours, within about 3 hours, within about 2 hours or within about 1 hour of the step of isolating the produced rAAV particles.

In embodiments, the production phase comprises adding sorbitol to the production phase medium. In embodiments, sorbitol is added to the production phase medium at one or more time points during the production phase, for example at about 6 hours, about 12 hours, about 20 hours, about 24 hours, about 48 hours after introduction of at least one of the first, second and third polynucleotide sequences (i.e., about 6 hours, about 12 hours, about 20 hours, about 24 hours, about 48 hours after beginning the production phase). In embodiments, the sorbitol is added to the production medium to a concentration of about 50 mM to about 200 mM, or about 80 mM to about 120 mM. In embodiments, the sorbitol is added to the production medium to a concentration of about 100 mM.

In embodiments, the production phase is at least about 48 hours (e.g., 47.5, 48, 50, 55, 60, 65, 70, 75, 80, 85, 90, 95, 96, 96.5, or 97 hours). In preferred embodiments, the production phase is about 72 to about 100 hours, about 90 hours to about 100 hours, about 92 hours to about 98 hours, or about 94 to about 98 hours. In an embodiment, the production phase is about 96 hours.

In embodiments of the method, the at least one of the first, second and third polynucleotide sequences are introduced into the cells by transfection of one or more vectors including the first, second and third polynucleotide sequences, by infection with one or more viral vectors including one or more of the first, second and third polynucleotide sequences, by a combination of transfection of the one or more vectors and infection of the one or more viruses, or by electroporation with the first, second and third polynucleotide sequences. In embodiments, at least one of the first, second and third polynucleotide sequences are introduced into the cells by transfection.

In embodiments of the method, the transgene encodes a therapeutic protein or a reporter protein. A non-exhaustive list of examples of transgenes includes RPGR, RPE65, GAD65, GAD67, and CNGB3. In some embodiments, the two AAV ITRs are AAV2 ITRs. In the method, the AAV cap gene can be from an AAV serotype or AAV variant such as, for example, AAV1, AAV2, AAV3, AAV4, AAV5, AAV6, AAV7, AAV8, AAV9, AAV10, AAV 11, AAV12, AAV13, AAVrh10, AAV-PHP.5, AAV-PHP.B, AAV-PHP.eB, AAV2-retro, AAV9-retro, and a hybrid thereof. In embodiments, the one or more helper genes can include all or part of one or more adenovirus genes, herpes simplex virus type 1 genes, or baculovirus genes.

In embodiments of the method, the cells are mammalian cells, e.g., HEK293 cells. The cells (e.g., HEK293 cells) can be adapted for culture in suspension. The at least one culture vessel can be one or more of, for example, a shaker flask, a spinner flask, a cellbag or a bioreactor. In embodiments, the culture medium for the expansion phase can have a pH of about 7.1 to about 7.5 (e.g., about 7.2 to about 7.4), and culturing the cells can include CO₂ sparging. In some embodiments, prior to the step of introducing at least one of the first, second and third polynucleotide sequences, the pH of the culture medium is allowed to drift between about 6.9 and about 7.3. In embodiments, prior to introducing into the cells one or more polynucleotide constructs, the pH of the culture medium is changed to about 6.9 and CO₂ sparging is halted.

In embodiments, isolating the rAAV particles comprises the step of lysing the cells after the production phase. In embodiments, the rAAV particles can be isolated at about 48 or more hours (e.g., 47.5, 48, 50, 55, 60, 65, 70, 75, 80, 85, 90, 95, 96, 96.5, 97 hours) after introduction of the first, second and/or third polynucleotide sequences (step (ii), above). For example, the rAAV particles can be isolated at about 90 to about 100 hours, about 92 hours to about 98 hours, or about 94 to about 98 hours after introduction of the first, second and third polynucleotide sequences. In embodiments, the rAAV particles are isolated (e.g., the cells are lysed) at about 96 hours after the introduction of the first, second and/or third polynucleotide sequences (e.g., about 96 hours after step (ii), above). In embodiments, the isolated rAAV particles are present in a lysate and the lysate is clarified using at least one filter resulting in a clarified lysate. In such embodiments, the method can further include subjecting the clarified lysate to one or more purification steps. Any suitable purification step or steps can be used, e.g., one or more of ultracentrifugation, affinity chromatography, and/or ion-exchange chromatography. In embodiments, the method provides at least about 40,000 to about 200,000 rAAV particles per cell, and/or: an average yield of greater than about 8×10¹² to about 1×10¹⁴ purified rAAV particles from 1 liter of transfected culture medium. In embodiments, the method provides a rAAV particle to empty AAV particle ratio of at least about 30%, e.g., about 30%-40%, at least 65%, about 65%-90%, etc. In embodiments, the method further includes determining at least one of: capsid concentration, viral genome titer, and rAAV particle to empty AAV particle ratio from a portion of the lysate or the clarified lysate. In embodiments of the method, the isolated rAAV particles are separated from empty AAV particles.

The present invention also provides a population of rAAV particles produced by the method, and a pharmaceutical composition including the population of rAAV particles.

### BRIEF DESCRIPTION OF THE FIGURES

FIG. 1A is a graph showing results from Ca²⁺ supplementation in a flask model for production of rAAV5 vectors containing a RPGR transgene. HEK293 cells were transfected with RPGR transgene plasmid, helper plasmid, and Rep/Cap plasmid, and 0.5 mM or 2 mM CaCl₂ was added at either 6 hours or 20 hours post transfection. Cells were lysed at either 72 hours or 96 hours post transfection. Vector genome (VG) and ratio of capsid full:empty (F:E) ratio for AAV5-RPGR in 72-hour and 96-hour post-transfection lysates are provided.

FIG. 1B is a graph showing results from Ca²⁺ supplementation in a flask model for production of rAAV2 vectors containing a GAD67 transgene. HEK293 cells were transfected with GAD67 transgene plasmid, helper plasmid, and Rep/Cap plasmid. At 20 or 24 hours post transfection, 100 mM sorbitol, 50 mM NaCl, 2 mM CaCl₂), 2 mM MgCl₂ or 2 mM CaCl₂)/MgCl₂ was added to the culture media. Cells were lysed at either 72 hours or 96 hours post transfection. Vector genomes (VG) of AAV2-GAD67 in 72-hour and 96-hour post-transfection lysates are provided.

FIGS. 2A and 2B are graphs showing rAAV2 production after Ca²⁺ supplementation, addition of anti-clumping agent (ACA), and/or addition of concentrated essential nutrients feeds during the production phase in a 250 mL bioreactor. FIG. 2A shows VG titre as VG/mL in lysate from a 96-hour harvest (post-transfection). FIG. 2B shows Full to Empty (F:E) ratios (percentages) in lysate from a 96-hour harvest (post-transfection).

FIGS. 3A and 3B are graphs showing effect on rAAV2 production from addition of different salts at 12 hours post-transfection. FIG. 3A shows VG titer as VG/mL and FIG. 3B shows F:E ratios (percentages).

FIGS. 4A and 4B are graphs showing effect on rAAV2 production from addition of CaCl₂) added to 2 mM at −1 to 24 hours post-transfection. The control was AAV2-no salt.

FIG. 4A shows VG titer as VG/mL and FIG. 4B shows F:E ratios (percentages).

FIGS. 5A and 5B are graphs showing the effect on rAAV2 production from addition of CaCl₂) to concentrations of 0 mM (control) to 10 mM. at 12 hours post-transfection. FIG. 5A shows VG titer as VG/mL and FIG. 5B shows F:E ratios (percentages).

FIGS. 6A and 6B are graphs showing the effect on rAAV2, rAAV5, or rAAV8 production of the addition of CaCl₂) to 2 mM at 12 hours post-transfection. FIG. 6A shows VG titer as VG/mL and FIG. 6B shows F:E ratios (percentages).

### DETAILED DESCRIPTION

The present disclosure provides methods for producing recombinant adeno-associated virus (rAAV). The methods for producing rAAV particles include the following steps:
(i) an expansion phase comprising increasing the number of cells in at least one culture vessel containing culture medium;
(ii) introducing into the cells a first polynucleotide sequence including a transgene flanked by AAV inverted terminal repeats (ITRs), and optionally a second polynucleotide sequence including AAV rep and cap genes, and/or a third polynucleotide sequence including one or more helper genes;
(iii) a production phase (in which rAAV particles are produced) comprising culturing the cells from step (ii) and adding calcium (Ca) ions to the culture medium at about 0 to about 24 hours after introduction of the first polynucleotide sequence such that the total concentration of Ca ions in the culture medium is greater than 0.3 mM and less than 10 mM; and
(iv) isolating the rAAV particles.

The present invention also provides a population of rAAV particles produced by the method, and a pharmaceutical composition including the population of rAAV particles.

In embodiments, the methods provide increased production titers and/or higher ratio of full to empty capsids (F:E). More specifically, the disclosure provides, e.g., a method for increasing the production titer and/or F:E ratio of rAAV by transfected cells by adding Ca ions (e.g., calcium salts such as CaCl₂)) to the production media. In embodiments, the production titer is increased by about 1.5 fold, about 1.8 fold, about 2 fold, about 3 fold, about 5 fold, about 10 fold or more compared to the production titer when no Ca ions are added to the production medium. In embodiments, the F:E ratio is increased by about 1.3 fold, about 1.5 fold, about 2 fold, about 5 fold, about 8 fold, about 10 fold, about 12 fold, or higher compared to the F:E ratio when no Ca ions are added to the production medium.

The methods are scalable to manufacturing scale, for example, cultures of about 5 to about 10, about 10 to about 20, about 20 to about 50, about 50 to about 100, about 100 to about 200 or more liters, and are applicable to rAAV comprising a wide variety of AAV serotypes/capsid variants.

rAAV vectors produced by the methods disclosed herein are useful for expressing a transgene in a target cell. These rAAV vectors may be used in gene therapy as they can introduce into a target cell a polynucleotide comprising a transgene that may be maintained and expressed in target cells. rAAV vectors are able to deliver heterologous polynucleotide sequences (e.g., polynucleotide sequences encoding a therapeutic protein or a reporter protein and regulatory elements for expression of the protein) to target cells in human patients. A non-exhaustive list of examples of transgenes includes RPGR, RPE65, GAD65, GAD67, and CNGB3. In some embodiments, the two AAV ITRs are AAV2 ITRs. In the method, the AAV cap gene can be from an AAV serotype or AAV variant such as, for example, AAV1, AAV2, AAV3, AAV4, AAV5, AAV6, AAV7, AAV8, AAV9, AAV10, AAV 11, AAV12, AAV13, AAVrh10, AAV-PHP.5, AAV-PHP.B, AAV-PHP.eB, AAV2-retro, AAV9-retro, and a hybrid thereof. In embodiments, the one or more helper genes can include all or part of one or more adenovirus genes, herpes simplex virus type 1 genes, or baculovirus genes.

The term “vector” refers to a vehicle for introducing a polynucleotide into a target cell. Vectors can be viral vectors (e.g., rAAV vector, HSV vector) or non-viral vectors such as plasmids, or DNA associated with compounds such as liposomes, gelatin, or polyamines. An expression vector is a vector that contains a polynucleotide sequence encoding a gene product (e.g., a protein or RNA) with the regulatory elements for expression in a host or target cell.

A “rAAV”, “rAAV vector”, “rAAV particle” or “rAAV virion” refer to a recombinant AAV vector genome packaged in (i.e., encapsidated by) capsid proteins for subsequent infection of a target cell, ex vivo, in vitro, or in vivo. These phrases exclude empty AAV capsids and AAV capsids lacking full recombinant AAV genome containing the transgene to be expressed in the target cell. Thus, a rAAV vector, in addition to the capsid, comprises a rAAV genome. A “rAAV genome” or “rAAV vector genome” refers to the polynucleotide sequence containing a transgene of interest that is ultimately packaged or encapsidated to form a rAAV particle. Typically, for rAAV, most of the AAV genome (including, e.g., the rep, cap, and aap genes) has been deleted, with one or both ITR sequences remaining as part of the rAAV genome along with the transgene. “Transgene” as used herein refers to a polynucleotide sequence encoding a gene product (for example, a therapeutic protein or reporter protein) and regulatory elements for expression of the gene product in a target cell.

“Empty capsids” and “empty particles” refer to AAV particles having an AAV capsid shell, but lacking in whole, or in part, the recombinant AAV genome comprising the transgene sequence and one or two ITRs. Such empty capsids do not function to transfer the transgene into a target cell or cells. In embodiments, the isolated rAAV particles are separated from empty AAV particles.

The rAAV genome (including, e.g., the ITRs) can be based on the same strain or serotype (or subgroup or variant), or they can be different from each other. As a non-limiting example, a rAAV plasmid or vector genome or particle (capsid) based upon one serotype genome can be identical to one or more of the capsid proteins that package the vector genome. In addition, a rAAV genome can be derived from an AAV genome (e.g., comprise one or more ITRs derived from the AAV2 genome) that are distinct from one or more of the capsid proteins that package the rAAV vector genome.

rAAV vectors that can be produced by the methods disclosed herein include any rAAV vectors comprising capsids and genomes derived from any AAV strain or serotype. As non-limiting examples, a rAAV vector capsid and/or genome can be based upon AAV1, AAV2, AAV3, AAV4, AAV5, AAV6, AAV7, AAV8, AAV9, AAV10, AAV11, AAV12, AAV13, AAV-PHP-5, AAV-PHP-B, AAV-PHP-eB, AAV2-retro, AAV9-retro, AAVrh74, AAVrh, AAVrh.10 (i.e., an AAV containing AAVrh.10 ITRs and AAVrh.10 capsid proteins), etc. In embodiments, the rAAV vector comprises a genome and capsid proteins derived from the same AAV strain or serotype. For example, the rAAV vector can be an rAAV2 vector (i.e., an rAAV containing AAV2 ITRs and AAV2 capsid proteins).

In embodiments, the AAV vector is a pseudotyped rAAV vector, containing ITRs from one AAV serotype and capsid proteins from a different AAV serotype. In some embodiments, the pseudotyped rAAV is rAAV2/5 (i.e., an rAAV containing AAV2 ITRs and AAV5 capsid proteins); rAAV2/8 (i.e., an rAAV containing AAV2 ITRs and AAV8 capsid proteins); rAAV2/9 (i.e., an AAV containing AAV2 ITRs and AAV9 capsid proteins) rAAV2/10 (i.e., an rAAV containing AAV2 ITRs and AAV10 capsid proteins). In embodiments, the rAAV vector comprises a capsid protein that is a variant AAV capsid such as the AAV2 variant rAAV2-retro (SEQ ID NO:44 from WO 2017/218842, incorporated herein by reference).

### Cell Lines

The rAAV vector production methods described herein generally require certain elements including, for example: (i) a permissive host cell for rAAV production (producer cell); (ii) helper virus functions which can be supplied, e.g., by a suitable construct containing genes providing adenoviral helper functions; (iii) a trans-packaging rep/cap construct; and (iv) suitable production media.

A producer cell is any cell that is a permissive host cell for production of rAAV once the rAAV genome construct, helper function construct, and construct providing AAV functions (e.g., expressing rep and cap) are present. The term can also include the progeny of the original cell which has been transfected. Thus, a producer cell is also a host cell which has been transfected with exogenous DNA sequence, or the progeny of the host cell where that DNA sequence has integrated into the host cell genome. It is understood that the progeny of a single parental cell may not necessarily be completely identical in morphology or in genomic or total DNA complement as the original parent, due to natural, accidental, or deliberate mutation.

In embodiments, cells used to produce rAAV particles are mammalian cells, including HEK293 cells, BHK cells, and HeLa cells. Exemplary producer/host cells include human embryonic kidney (HEK) cells such as HEK293. In preferred embodiments, the producer cells are adapted for growth in suspension, including suspension adapted HEK293 cells. In further preferred embodiments, the producer cells are adapted for growth in serum-free medium. In embodiments, the producer cells are increased in at least one culture vessel which can be one or more of, for example, a shaker flask, a spinner flask, a cellbag or a bioreactor.

Producer cell lines that can be used in the rAAV production methods disclosed herein include mammalian or insect cell lines. The term “cell line” refers to a population of cells capable of continuous or prolonged growth and division in vitro under appropriate culture conditions. Cell lines can, but need not be, clonal populations derived from a single progenitor cell. In cell lines, spontaneous or induced changes can occur in karyotype during storage or transfer of such clonal populations, as well as during prolonged passaging in tissue culture. Thus, progeny cells derived from the cell line may not be precisely identical to the ancestral cells or cultures.

For rAAV production to occur, the producer cell line may require that one or more of the rAAV genome production construct, helper function construct, and/or AAV rep/cap construct are present within the producer cell. These may be introduced as three constructs (e.g., three plasmids) or the producer cells may already have one or more constructs providing some or all of these functions stably integrated into the producer cell genome. As used herein, the term “stable” in reference to a cell, or “stably integrated” means that the nucleic acid sequences, such as a selectable marker and/or heterologous nucleic acid sequence, or plasmid or vector (or portion thereof) has been inserted into a chromosome (e.g., by homologous recombination, non-homologous end joining, transfection, etc.) or is maintained in the recipient cell or host organism extrachromosomally, and has remained in the chromosome or is maintained extrachromosomally for a period of time.

### Expanding the Producer Cell Line

In embodiments of the method described herein, an expansion phase or expansion step is used to increase the number of producer cells prior to the step of introducing the rAAV genome production construct (containing the rAAV genome) and/or other constructs providing helper virus functions and AAV functions. The expansion phase or expansion step may be performed in one or more cell culture vessels. For example, the expansion phase or expansion step may be performed in a series of cell culture vessels of increasing volume. The cell culture medium used for expansion of the producer cell line can be any medium appropriate for the growth (i.e., increase in number) of the producer cells. In preferred embodiments, the expansion phase culture media is animal component free, and does not include, for example serum or other components derived from animals. Chemically defined, animal component-free media is commercially available.

In embodiments, an anticlumping supplement, at times referred to herein as anti-clumping agent (ACA), is added to the expansion medium to reduce cell aggregation. ACA is commercially available from, e.g., Irvine Scientific. The anti-clumping supplement may be added at one or more time points to the expansion phase culture media. In embodiments, the anti-clumping supplement comprises dextran sulfate, heparin and/or other sulfated glycosaminoglycans that suppress the aggregation of the producer cells. In embodiments, the anticlumping supplement comprises sodium heparin, which can be added to the media to concentrations of about 25 μg/ml to about 250 μg/ml, for example, about 25 μg/ml, about 50 μg/ml, about 100 μg/ml, about 150 μg/ml, and/or about 200 μg/ml.

In embodiments, the expansion phase culture media comprises, and/or is supplemented to comprise, one or more of glutamine, a glutamine precursor or an amino acid dipeptide including glutamine at concentrations from about 2 mM to about 6 mM (e.g., about 2 mM, about 3 mM, about 4 mM, about 5 mM or about 6 mM). The one or more of glutamine, glutamine precursor or an amino acid dipeptide including glutamine can be one or more of, e.g., L-alanyl-L-glutamine, L-glutamine, glutamate, glycyl-L-glutamine, glutamine protein hydrolysate, L-glutamic acid, and a glutamine dipeptide. A commercially available example of a glutamine supplement provided as the dipeptide L-alanyl-L-glutamine is GlutaMAX (ThermoFisher).

In embodiments, the expansion phase culture media comprises, and/or is supplemented to comprise, a non-ionic polyol surface-active agent such as poloxamer 188 (a copolymer of polyethylene and polypropylene ether glycol). In embodiments, the non-ionic polyol surface-active agent is present in the expansion phase culture media at about 0.05% to about 0.2% (w:v) (e.g., about 0.05%, about 0.1%, about 0.1%, or about 0.2%). In embodiments, the expansion phase culture media comprises about 4 mM L-alanyl-L-glutamine dipeptide and 0.1% (w:v) poloxamer 188.

In embodiments, the pH of the expansion phase culture media is maintained at a pH of about 7.1 to about 7.5 (e.g., about 7.1, about 7.2, about 7.3, about 7.4, or about 7.5). In embodiments, the pH is maintained at about 7.2 to about 7.4 by CO₂ sparging. In embodiments, prior to introducing into the cells one or more polynucleotide constructs, the pH of the culture medium is changed to about 6.9 and CO₂ sparging is halted.

### Introducing One or More Polynucleotide Constructs

rAAV vector generation typically requires a production cell line that provides the basic biosynthetic machinery, as well as (i) a construct that provides the rAAV genome (the transgene of interest and associated regulatory elements flanked by AAV ITRs) and (ii) one or more constructs with additional genes that provide the gene products needed to direct rAAV vector production. These additional genes include AAV-derived genes (e.g., AAV rep and cap) and helper virus-derived genes (e.g., adenovirus E1a, E1b, E2a, E4 and VA) required to support vector genome replication and packaging.

In embodiments, at least one of the first, second and third polynucleotide sequences are introduced into the cells by transfection of one or more vectors including the first, second and third polynucleotide sequences, by infection with one or more viruses including one or more of the first, second and third polynucleotide sequences, by a combination of transfection of the one or more vectors and infection of the one or more viruses, or by electroporation with the first, second and third polynucleotide sequences.

“Helper virus genes” or “helper virus-derived genes” refers to non-AAV derived viral genes whose gene products AAV is dependent on for replication. The term includes proteins and/or RNAs that are required in AAV replication, including those involved in activation of AAV gene transcription, stage specific AAV mRNA splicing, and AAV DNA replication. Helper virus genes can be derived from any of the known AAV helper viruses such as adenovirus, herpesvirus and vaccinia virus. Thus, “helper virus functions” refers to those functions provided by helper virus genes that are required for AAV production (e.g., adenovirus E1a, E1b, E2a, E4 and VA). These helper virus functions can be provided on one or more vectors introduced into the producer cells, stably expressed by the producer cells, or a combination of both.

As used herein, “AAV functions,” or “AAV accessory functions” refer to AAV-derived coding sequences which can be expressed in producer cells to provide AAV gene products that function in trans for productive AAV replication and packaging. Thus, AAV functions include AAV open reading frames (ORFs), including rep and cap and others such as aap for certain AAV serotypes. Such AAV functions are provided by one or more polynucleotide constructs, which can be plasmid vectors, non-plasmid vectors, or a polynucleotide construct that has been integrated into a chromosome of the producer cell, that provides AAV helper functions. Plasmids that provide AAV functions that may be used in the methods disclosed herein are commercially available.

In embodiments of the method, one or more of the helper virus genes are constitutively expressed by producer cells (e.g., HEK293 cells), while other helper virus genes are introduced into the producer cells, e.g., by transfection of one or more polynucleotide constructs encoding the remaining helper virus genes needed for AAV production. The AAV-derived genes (e.g., rep and cap) may be included in the same polynucleotide construct containing one or more helper virus genes, or may be on a separate polynucleotide construct. rAAV particles are produced after a polynucleotide construct comprising a rAAV genome (e.g., a rAAV genome production vector) is introduced into the producer cell line. In embodiments, the rAAV particles are produced after transiently transfecting producer cells with (i) an rAAV genome production vector, and (ii) one or more vectors that provide helper virus genes (e.g., E4, E2a, and VA) and AAV genes (e.g., rep and cap). In embodiments these vectors are plasmids.

In embodiments of the method disclosed herein, following the expansion phase a first polynucleotide construct comprising a transgene flanked by ITRs, and a second polynucleotide construct comprising helper virus genes and AAV rep and cap genes are introduced into the expanded producer cells. When the first and second polynucleotide constructs are plasmids, this system may be referred to as a two-plasmid system.

In embodiments of the method disclosed herein, following the expansion phase a first polynucleotide construct comprising a transgene flanked by ITRs, a second polynucleotide construct comprising helper virus genes, and a third polynucleotide construct comprising AAV rep and cap genes are introduced into the expanded producer cells. When the first, second and third polynucleotide constructs are plasmids, this system may be referred to as a three-plasmid system.

In cases where one or more recombinant plasmids are used to manufacture rAAV vectors, the “rAAV genome production plasmid” refers to a plasmid comprising the transgene (operably linked to regulatory sequences) and one or more ITRs intended for packaging into the rAAV, as well as non-rAAV genome components (the plasmid backbone) that are important for cloning and amplification of the plasmid, but are not packaged or encapsidated into rAAV vectors.

The terms “transduce” and “transfect” refer to introduction of a polynucleotide into a host cell or target cell. In embodiments, the host cell is a producer cell, e.g., a HEK293 cell. In embodiments, the rAAV genome production plasmid along with one or more plasmids providing helper virus functions and AAV functions are introduced into the producer cells by transient transfection methods. The transient transfection of producer cells to introduce the first polynucleotide construct comprising a transgene and ITR(s) (e.g., an rAAV genome production vector); and optionally a second and/or third polynucleotide constructs providing AAV functions (rep and cap genes), and helper virus functions, can be accomplished by standard transfection methods including for example calcium phosphate coprecipitation, cationic lipid-based transfection, and cationic polymer-based transfection. Cationic lipid-based transfection includes e.g., Lipofectamine (a 3:1 mixture of DOSPA (2,3-dioleoyloxy-N-[2(sperminecarboxamido)ethyl]-N,N-dimethyl-1-propaniminium trifluoroacetate) and DOPE (1,2-Dioleoyl-sn-glycero-3-phosphoethanolamine). Cationic polymer-based transfection includes, e.g., using linear and/or branched polyethylenimine (PEI), poly-L-lysine, poly-L-arginine, polyamidoamine dendrimers and others. In embodiments the transient transfection of producer cells is performed using a PEI based transfection reagent. The PEI may be a linear or branched polymer. In embodiments, the PEI is a 20-25 kD linear PEI. For example, in embodiments the PEI is jetPEI or PEIpro (available from Polyplus). Additionally, transient transfection of producer cells can be performed using a transfection reagent comprising both cationic lipids and cationic polymers.

rAAV may alternatively be produced in insect cells (e.g., sf9 cells) using baculoviral vectors or in HSV-infected baby hamster kidney (BHK) cells (e.g., BHK21). In both methods, rAAV production is triggered in the host cells, insect cells or mammalian cells, respectively, upon co-infection with two or more recombinant viruses carrying the rAAV genome and one more AAV rep and cap, and helper virus functions required for rAAV replication and packaging.

Producing the rAAV Particles

The methods disclosed herein include a production phase (also referred to as a production step) after the step introducing a rAAV genome vector and/or vectors providing helper virus functions and/or AAV functions into the producer cells. In the methods described herein, rAAV particles are produced by culturing the cells following introduction of the rAAV genome vector for at least about 48 hours (e.g., 47.5, 48, 50, 55, 60, 65, 70, 75, 80, 85, 90, 95, 96, 96.5, or 97 hours). In embodiments, the transfected producer cells are cultured (i.e., the production phase is maintained) for about 72 to about 100 hours, about 90 hours to about 100 hours, about 92 hours to about 98 hours, or about 94 to about 98 hours. In embodiments the production phase is maintained for about 96 hours.

The production phase medium can be any cell culture medium suitable for production of rAAV in the producer cells. In embodiments, the production medium is free of animal products, such as serum. “Free of” in this context means that the medium has undetectable levels of animal products such as serum. In embodiments, the pH of the production medium is reduced compared to the pH of the expansion phase medium. In embodiments, the production medium is maintained at a pH of about 6.8 to about 7.4 (e.g., about 6.8, about 6.9, about 7.0, about 7.1, about 7.2, about 7.3, or about 7.14). In embodiments the pH is maintained at about 6.9 to about 7.3.

In embodiments of the method, the production phase comprises addition of calcium ions to the production phase cell culture medium. Conventionally in the art, levels of calcium ions are typically maintained at low levels (usually around 0.1 mM) in media formulations used in suspension cell culture processes to reduce or prevent cell aggregation. Proteins involved in cell-cell adhesion such as E-cadherins are calcium dependent. The extracellular domain of E-cadherins forms a calcium dependent homophilic trans-dimer, providing specific interaction with adjacent cells, while the cytoplasmatic domain is connected to the actin cytoskeleton. Therefore, in the art it is generally considered desirable to maintain low levels of calcium.

Addition of calcium ions to the production medium, also referred to herein as calcium supplementation, comprises adding calcium ions (Ca²⁺) in the form of calcium salts such as CaCl₂). Calcium ions can be added during the production phase at one or more times following introduction of the rAAV genome vector (e.g., post-transfection). For example, calcium ions can be added at one or more times between about 0 hours to about 48 hours from the start of the production phase (i.e., post transfection), e.g., about 1 hour, about 6 hours, about 10 hours, about 12 hours, about 20 hours, about 24, hours about 30 hours, about 36 hours, and/or about 48 hours. In embodiments, calcium ions are added to the production media at one or more times between about 0 hours to about 24 hours, about 0 hours to about 20, about 0 hours to about 18 hours, or about 6 hours to about 12 hours after the start of the production phase (i.e., post-transfection). In embodiments, calcium ions are added to the production phase medium at about 6 hours post transfection.

Calcium ions can be added to the production media to achieve a total concentration of calcium ions in the culture medium greater than 0.3 mM and less than 10 mM. In embodiments, calcium ions are added to a total concentration between about 1 mM to about 9 mM, between about 1 mM to about 8 mM, between about 1 mM to about 7 mM, between about 2 mM to about 9 mM, between about 2 mM to about 8 mM, between about 2 mM to about 7 mM, between about 2 mM to about 6 mM, between about 2 mM to about 5 mM, or between about 2 mM to about 4 mM. In embodiments, calcium ions (e.g., CaCl₂)) is added to a concentration of about 1 mM, about 1.5 mM, about 2 mM, about 2.5 mM, about 3 mM, about 3.5 mM, about 4 mM, about 4.5 mM, about 5 mM, about 5.5 mM, about 6 mM, about 6.5 mM, about 7 mM, about 7.5 mM, about 8 mM, about 8.5 mM, about 9, or about 9.5.

In embodiments, the production phase comprises adding one or more of glutamine, a glutamine precursor or an amino acid dipeptide including glutamine to the production phase medium. The one or more of glutamine, glutamine precursor or an amino acid dipeptide including glutamine can be one or more of, e.g., L-alanyl-L-glutamine, L-glutamine, glutamate, glycyl-L-glutamine, glutamine protein hydrolysate, L-glutamic acid, and a glutamine dipeptide. A solution including at least one of glutamine, glutamine precursor or an amino acid dipeptide including glutamine can be added to the production phase culture medium at, e.g., one or more of about 6 hours, about 12 hours, about 24 hours, about 48 hours or about 72 hours post-transfection.

In embodiments, the production phase comprises adding sorbitol to the production phase medium. The sorbitol can be added to the production phase medium at one or more time points during the production phase, for example at about 6 hours, about 12 hours, about 20 hours, about 24 hours, and/or about 48 hours post transfection. In embodiments, the sorbitol is added to the production medium to a concentration of about 50 mM to about 200 mM, or about 80 mM to about 120 mM. In embodiments, the sorbitol is added to the production medium to a concentration of about 100 mM.

In embodiments, the production phase comprises addition of an anti-clumping supplement to the production phase medium. The anti-clumping supplement may be added at one or more time points to the production phase culture media (for example, at one or more of about 6, about 10, about 12, about 20, about 24, about 48 or about 72 hours post-transfection). In embodiments, the anti-clumping supplement comprises dextran sulfate, heparin and/or other sulfated glycosaminoglycans that suppress the aggregation of the producer cells. In embodiments, the anticlumping supplement comprises sodium heparin, which can be added to the media to concentrations of about 25 μg/ml to about 250 μg/ml, for example, about 25 μg/ml, about 50 μg/ml, about 100 μg/ml, about 150 μg/ml, and/or about 200 μg/ml.

In embodiments, anti-clumping supplement is not added to the production phase media, or is only added to the production phase media shortly before the end of the production phase, for example within about 24 hours, about 12 hours, within about 6 hours, within about 3 hours, within about 2 hours, or within about 1 hour of the end of the production phase.

Isolating the rAAV

Embodiments of the methods described herein include isolating the rAAV particles at the end of the production phase. In embodiments, the rAAV particles can be isolated at about 48 or more hours (e.g., 47.5, 48, 50, 55, 60, 65, 70, 75, 80, 85, 90, 95, 96, 96.5, 97 hours) after introduction of the first, second and/or third polynucleotide sequences (after step (ii), above, in other words after the start of the production phase). For example, the rAAV particles can be isolated at about 90 to about 100 hours, about 92 hours to about 98 hours, or about 94 to about 98 hours after introduction of the first, second and third polynucleotide sequences. In embodiments, the rAAV particles are isolated (e.g., the cells are lysed) at about 96 hours after the introduction of the first, second and/or third polynucleotide sequences (e.g., about 96 hours after step (ii), above). Isolating the rAAV can include multiple steps including, for example, lysing the producer cells, clarifying the lysate, and subsequent purification steps.

rAAV particles may be retained within producer cells following generation, and methods to release intracellular rAAV vector include physical and chemical disruption, for example, use of detergent, microfluidization and/or homogenization. Concurrently during cell lysis and/or subsequently after cell lysis, a nuclease such as benzonase may be added to degrade contaminating DNA.

Typically, the resulting lysate is clarified to remove cell debris and provide a clarified cell lysate. Filtration technologies are useful to separate rAAV vector from larger particles such as cell debris (microfiltration) as well as from smaller molecules such as soluble protein impurities (ultrafiltration). For example, rAAV vector-containing cells that have been disrupted can be passed through one or more micron diameter pore size filters (such as a 0.1-10.0 μm pore size filter, for example, a 0.45 μm and/or pore size 0.2 μm filter). A two-stage 0.45/0.2 μm cartridge filter can be used to separate rAAV vectors, that are recovered in the filter permeate, from cell debris, which is retained by the filter. In embodiments, the method further includes determining at least one of: capsid concentration, viral genome titer, and rAAV particle to empty AAV particle ratio from a portion of the lysate or the clarified lysate.

The rAAV vectors in the clarified lysate can be further isolated (purified) by use of one or more additional purification methods including ultracentrifugation, affinity chromatography, ion-exchange chromatography, and tangential flow filtration (TFF).

In embodiments, the isolated rAAV particles are separated from empty AAV particles, and the method provides a rAAV particle to empty AAV particle ratio of at least about 30%, e.g., about 30%-40%, at least 65%, about 65%-90%, etc. Levels of nuclease-resistant, AAV-encapsidated DNA impurities can be assessed by qPCR using primers and probes designed for relevant sequences in helper plasmids, or to high copy number genomic sequences. Sensitivity to nuclease treatment performed prior to qPCR allows distinction between nuclease-sensitive ‘naked’ residual DNA impurities and nuclease-insensitive-encapsidated residual DNA impurities. Total AAV capsids can be measured using capsid-specific ELISA assays and the amount of empty capsid determined by comparison of the capsid particle titer and the VG titer. Spectrophotometric methods can be used for samples in which non-AAV capsid impurities have been substantially removed.

### Examples

### Example 1. Evaluation of Calcium Chloride Addition on AAV Production in a Shaker Flask Model

Calcium chloride addition at different production time points and concentrations, as well as production length, was evaluated in suspension adapted HEK293 cells cultured in E125 shaker flasks. At the end of the flask process, cultures were harvested and chemically lysed. Performance between conditions was evaluated based on physical capsid and virus genome titer.

### Seed Train and Expansion:

HEK293 cells were expanded to a sufficient number for the study in Erlenmeyer flasks (E1000) and cultured in a CO₂ shaking incubator (New Brunswick S41i). Seed train flasks were inoculated at seeding density of 2×10⁵ viable cells (VC) per mL in a seed volume of 200 ml. The seed medium was chemically defined medium supplemented with 4 mM GlutaMAX and 0.1% w:v Poloxamer 188. Forty-eight hours after seeding 180 mL feed chemically defined medium was added (9:10 feed:seed medium volume ratio).

Anti-clumping agent (ACA, Fujifilm Irvine Scientific) was added at 1 mL/L during the first passage and periodically thereafter, in order to control cell aggregation.

HEK293 cells were cultured at 37° C. with CO₂ at 8% and agitation at 150 rpm with 1 inch orbital rotation. Cells were cultured in chemically defined medium supplemented with 4 mM GlutaMAX and 0.1% w:v Poloxamer 188.

### Transfection Conditions for AAV5-RPGR:

HEK293 cells in E1000 non-baffled flasks (Corning) were transfected 96 h post-inoculation at cell density of about 1.8-2.4×10⁶ VC/mL with plasmids: Helper (pFD6k, 15.4 kb); Rep/Cap (pRC2-5k, 8.1 kb); Transgene (pRPGR-k, 7.7 kb) at a plasmid copy number ratio of 1:1:2 with total DNA quantity of 0.95 μg total DNA per 10⁶ viable cells at transfection.

The transfection mixture was prepared with a PEI:DNA ratio of 1.3:1 (v:w) with a DNA concentration of 30 mg/L. Medium for the transfection mixture was chemically defined culture medium (not supplemented). The Helper, Rep/Cap and Transgene constructs were added to culture medium. PEI was added to medium, mixed, and then the mixture was added to the DNA mix followed by rotation and incubation for 15 min. After the 15 min incubation, the transfection mix was added to the flasks, which were returned to the shaker incubator.

Transfection Conditions for rAAV2-GAD67:

HEK293 cells in E1000 non-baffled flasks (Corning) were transfected 96 h post-inoculation at cell density of about 1.8-2.4×10⁶ VC/mL with plasmids: Helper (pFD6k, 15.4 kb); Rep/Cap (pNLRep2-2-K, 10.3 kb); Transgene (pGAD67k, 7.1 kb) at a plasmid copy number ratio: 2:1.5:1 with total DNA quantity of 1.0 μg total DNA/10⁶ viable cells at transfection.

The transfection mixture was prepared with a FectoVIR:DNA ratio of 1.5:1 (v:w) with a DNA concentration of 80 mg/L. Medium for the transfection mixture was chemically defined culture medium (not supplemented). The Helper, Rep/Cap and Transgene constructs were added to the medium. FectoVIR was added to culture medium, mixed, and then added to the DNA mix followed by rotation and incubation for 15 min. After the 15 min incubation, the transfection mix was added to the flasks, which were returned to the shaker incubator.

rAAV Production Conditions:

For HEK293 cells transfected with AAV5-RPGR plasmid, one hour post-transfection cells were spit into E125 non-baffled flasks (Corning) and incubation continued at 37° C., 8% CO₂, 150 RPM, 1″ orbital rotation. Transfected HEK293 cells were incubated at 37° C., 8% CO₂, 180 RPM, 1″ orbital rotation and 1 M CaCl₂) stock solution was added to a concentration of 0.5 mM or 2.0 mM at either 6 hours or 24 hours post-transfection. For control flasks no stock solution was added.

For HEK293 cells transfected with AAV2-GAD67 plasmid, one hour post-transfection cells were spit into E125 non-baffled flasks (Corning) and incubation continued at 37° C., 8% CO₂, 150 RPM with 1″ orbital rotation. Transfected HEK293 cell were incubated at 37° C., 8% CO₂, 150 RPM with 1″ orbital rotation and one of the following was added: 20 hours post transfection 4 M D-sorbitol to a concentration of 100 mM sorbitol; 20 hours post transfection 4 M NaCl stock solution to a concentration of 50 mM NaCl; 24 hours post transfection 1 M CaCl₂) stock solution to a concentration of 2 mM CaCl₂); 24 hours post transfection 1 M MgCl₂ stock solution to a concentration 2 mM MgCl₂; or 24 hours post transfection 1 M CaCl₂) and 1 M MgCl₂ stock solutions to a concentration 2 mM CaCl₂) and 2 mM MgCl₂. For control flasks no stock solution was added.

### Lysis Conditions:

Cells were harvested 96 h post transfection. 50X Lysis buffer (formulated to achieve final concentration of: 0.1% Triton X-100, 2 mM MgCl₂, 1 mM Tris, 125 U/10⁷ cells of Benzonase) was added at 2% v/v to cell culture and incubated for 2 hours at 37° C. and 150 RPM.

### Determination of Viral Capsid and Viral Genome Titre:

Viral capsid (VC) titre was determined by ELISA using AAV Titration ELISA Kits from Progen™ or GYROLAB® AAVX Titer kits from Gyros Protein Technologies, by following the manufacturer's guidelines.

Viral genome (VG) concentration and titer was determined by qPCR. Samples were subjected to a DNAse I treatment followed by a Proteinase K treatment. Four serial dilutions were assayed by mixing an adequate amount of sample with qPCR master mix containing nucleotides, forward primer, reverse primer and a Taqman probe targeted to a specific region of each transgene. A seven-point standard curve was established using plasmid DNA or a DNA oligonucleotide containing the targeted region and was used to assess the number of viral genomes per sample.

### Results:

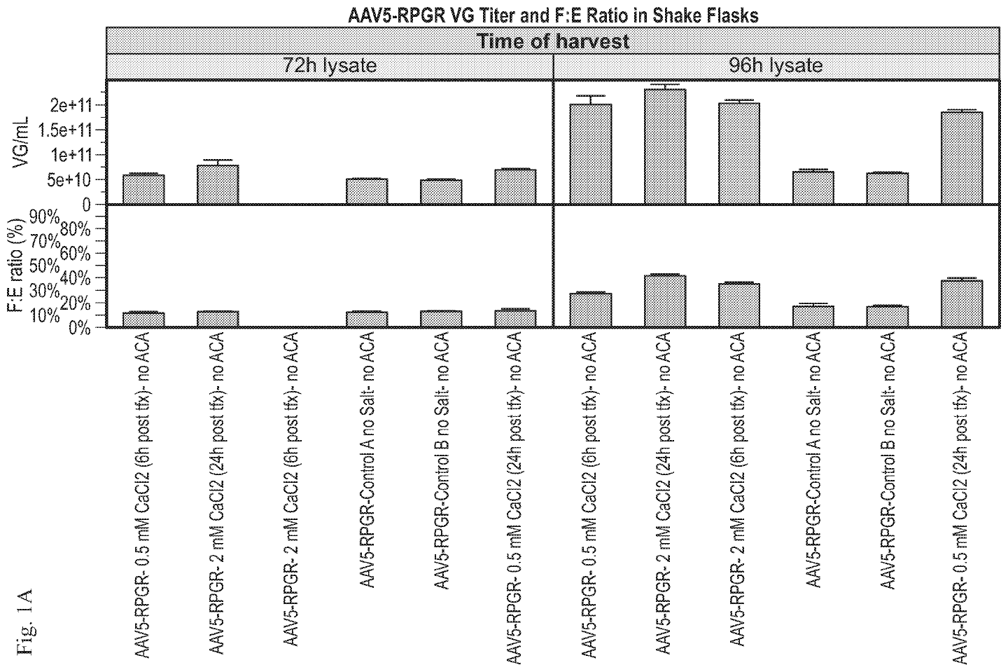

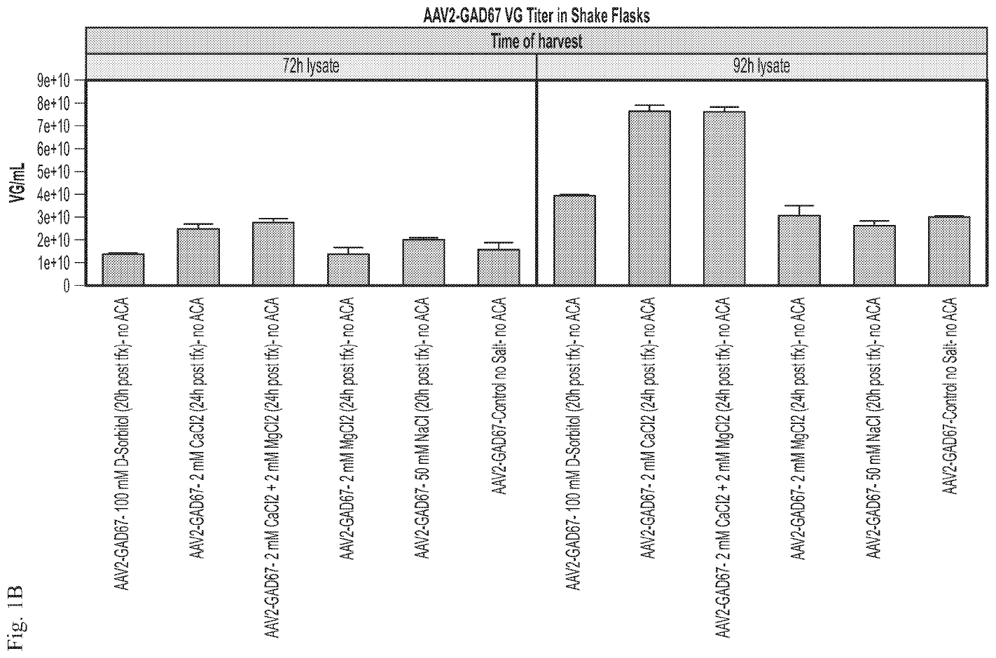

Viral genome (VG) titer was increased 3.5 fold for rAAV5 after addition of 0.5 mM or 2 mM CaCl₂) at 6 or 20 hours post-transfection compared to control cultures without supplementation. See FIG. 1A. Ca²⁺ supplementation increased viral genome (VG) titer by about 4 to 5 fold for rAAV2 compared to control cultures without supplementation. See FIG. 1B. In addition, full to empty capsid (F:E) ratio increased from 10-15% in the control cultures to 30-40% for rAAV5. See FIG. 1A. Viral genome titer and F:E ratio were both significantly enhanced when Ca²⁺ supplementation was combined with production times extended to 96 hours post transfection. See FIGS. 1A and 1B. Supplementation of NaCl or MgCl₂ did not increase VG titer compared to the control, while supplementation with sorbitol appears to provide some improvement in VG titer of rAAV2 when harvested at 96 hours post-transfection. See FIG. 1B.

### Example 2: Evaluation of Calcium Chloride Addition on AAV Production in a Bioreactor at 250 ml Scale

This study uses stirred tank bioreactors at 250 mL scale to evaluate addition of CaCl₂), time of addition of CaCl₂), ACA addition, addition of GlutaMAX, and addition of certain feeds during the AAV production phase.

Seed train: Suspension adapted HEK293 cells were expanded to a sufficient number for the study in Erlenmeyer flasks (E125, E250, E500 and E1000) and cultured in a CO₂ shaking incubator (New Brunswick S41i). Seed train flasks were inoculated at seeding density of at either 1.5 or 3.0×10⁵ VC/mL and expanded for 96 h and 72 h culture time, respectively.

Anti-clumping agent (ACA, Irvine Scientific) was added at 1 mL/L during the first passage and periodically thereafter in order to control cell aggregation.

HEK293 cells were cultured at 37° C. with CO₂ at 8% and agitation at 150 rpm with 1 inch orbital rotation. Cells were cultured in chemically defined medium supplemented with 4 mM GlutaMAX and 0.1% w:v Poloxamer 188.

### Bioreactor Cell Expansion

The seed medium for expansion in the 250 mL stirred tank bioreactor was chemically defined medium supplemented with 4 mM GlutaMAX and 0.1% w:v Poloxamer 188. HEK293 cells were seeded at a target seeding density of 2.0×10⁵ VC/mL in a volume of 45% of total working volume (113 mL).

At 48 hours post-inoculation, cells were fed with chemically defined medium, non-supplemented, with a feed volume of 40% of total working volume (100 mL).

Temperature setpoint was 37° C., pH setpoint was 7.30±0.05, and DO setpoint was 50%. DO level was controlled through the sparging of air and pure oxygen in the bioreactor, and pH was controlled through the addition of CO₂ (acid) and sodium hydroxide (base). Agitation was varied between 7.5 W/m³ at 45% total working volume and 17.5 W/m³ at 85% total working volume.

### Transfection

HEK293 cells were transfected 96 h post-inoculation at cell density of about 1.6-2.2×10⁶ VC/mL with plasmids: Helper (pFD6k, 15.4 kb); Rep/Cap (pNLRep2-2-K, 10.3 kb); Transgene (pGAD67k, 7.1 kb) at a plasmid copy number ratio: 2:1.5:1 with total DNA quantity of 1.0 μg total DNA/10⁶ viable cells at transfection.

The transfection mixture was prepared with a FectoVIR:DNA ratio of 1.5:1 (v:w) and a DNA concentration of 80 mg/L in the transfection mixture. Medium for the transfection mixture was chemically defined culture medium (not supplemented). The Helper, Rep/Cap and Transgene constructs were added to the medium. FectoVIR was added to culture medium, mixed, and then added to the DNA mix followed by rotation and incubation for 15 min. After the 15 min incubation, the transfection mix was added to the bioreactor.

rAAV Production

rAAV production parameters: temperature set to 37° C.; DO setpoint 50%; pH setpoint 6.9, no CO₂ addition; and agitation setpoint 17.5 W/m³ and 85% total working volume. The production conditions tested are provided in Table 1.

For conditions where feed was added, BalanCD HEK293 feed was added 4 times (at the first calcium addition, 24 h, 48 h and 72 h post-transfection) during the production phase at 3% v/v per addition (6.4 mL per addition). In runs where CaCl₂) was added, a 1 M stock solution of CaCl₂) was added to a concentration of 2 mM CaCl₂) at either 6, 12 or 20 hours post-transfection. In runs where ACA was added, 2 mL/L ACA was added 20 hours post-transfection. In runs where GlutaMAX was added during the production phase, GlutaMAX was added 4 times (at 6, 24, 48 and 72 h post-transfection) to achieve a concentration of 1 mM of GlutaMAX per addition in the bioreactor (1.1 mL per addition). When adding CaCl₂), ACA, BalanCD HEK293 Feed, or GlutaMAX at the same timepoint, individual solutions were combined.

TABLE 1
Bioreactor rAAV production conditions tested.

| | | CaCl₂ addition (hrs | BalanCD | |
|---|---|---|---|---|
| ID No.: | ACA | post-transfection) | HEK293 Feed | GlutaMAX |
| 1 | Yes | 6 | No | No |
| 2 | Yes | 12 | No | No |
| 3 | Yes | 20 | No | No |
| 4 | No | 6 | No | No |
| 5 | No | 12 | No | No |
| 6 | No | 20 | No | No |
| 7 | Yes | 12 | Yes | Yes |
| 8 | Yes | 12 | Yes | No |
| 9 | Yes | 12 | No | Yes |
| 10 | No | 12 | Yes | Yes |
| 11 | No | 12 | Yes | No |
| 12 | No | 12 | No | Yes |
| 13 (Control) | Yes | N/A | No | No |
| 14 (Control) | Yes | N/A | No | No |

### Cell Lysis

Cells were harvested 96 h post transfection. 50X Lysis buffer (formulated to achieve final concentration of: 0.1% Triton X-100, 2 mM MgCl₂, and 1 mM Tris) was added at 2% v/v to the bioreactor along with Benzonase 125 U/10⁷ total cells and incubated for 2 hours at 37° C. and 17.5 W/m3 at 85% total working volume.

### Determination of Viral Capsid and Viral Genome Titre

Determination of viral capsid and viral genome titer performed as described above.

### Results:

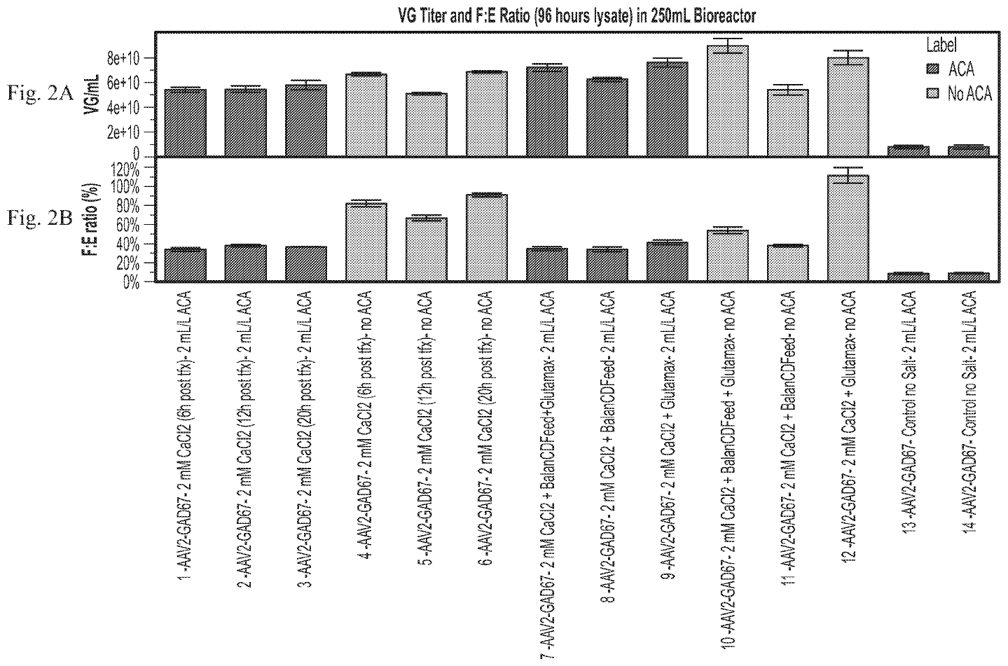

Calcium supplementation during the rAAV production phase in a bioreactor scale down model increased VG titre by 7 to 8 fold for rAAV2 compared to control and increased F:E ratio from 5-10% to 65-90%. See FIGS. 2A and 2B. Addition of ACA and glucose-based feeds during the production phase in addition to Ca²⁺ supplementation reduced the F:E ratio compared to Ca²⁺ supplementation without ACA or glucose-based feed. However, the F:E ratio was still about four fold higher than controls. See FIG. 2B. Glutamine supplementation appears to improve VG titre compared to runs with the same supplementation (including CaCl₂)) during production phase but without glutamine. See FIG. 2A.

### Example 3: Evaluation of Timing and Concentration of Salt Addition on rAAV Production

Production of rAAV2 and rAAV5 serotypes was evaluated by examining VG titre and F:E capsid ratio after addition of CaCl₂) at different time point or at different concentrations, as well as evaluating the effects of adding other salts. Timing of addition of CaCl₂) (to a final concentration of 2 mM) was evaluated with CaCl₂) added 1 hour before transfection (−1 hour), at transfection (0 hours), or 6 hours, 12 hours, 18 hours and 24 hours post-transfection. A range of CaCl₂) concentrations were tested with CaCl₂) added to final concentrations of 0.3 mM, 2 mM, 4 mM, 6 mM, or 10 mM at 12 hours post-transfection. In addition, addition of CaCl₂) to a final concentration of 2 mM was compared to addition of MgCl₂ to a final concentration of 2 mM or NaCl to a final concentration of 4 mM each added 12 hours post-transfection.

Methods for seed train and expansion, transfection, production, and lysis were substantially the same as those described above for rAAV2-GAD67 and rAAV5-RPGR, except ACA was not added post-transfection to any of the conditions. Production phase was ended and cells were lysed at 96 hours post-transfection.

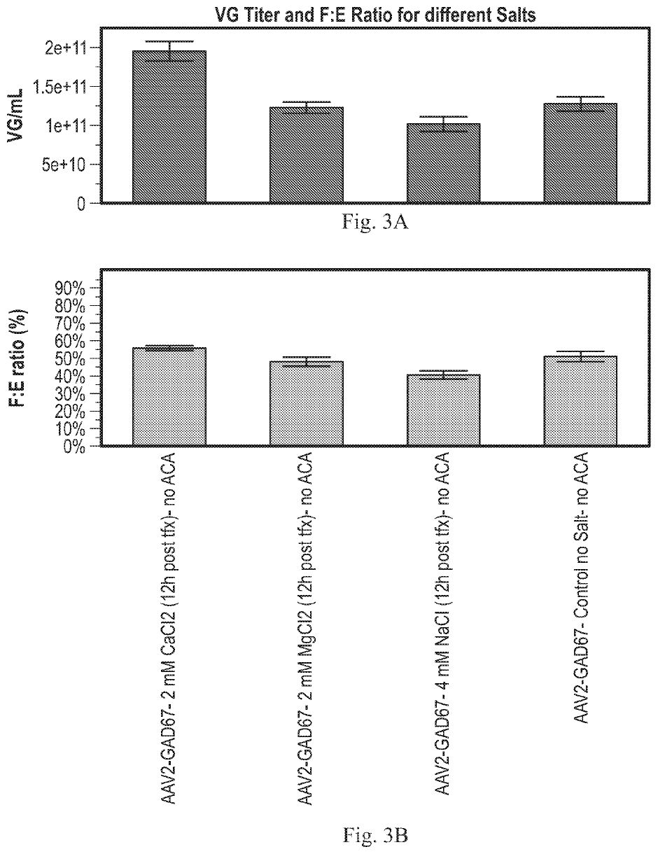

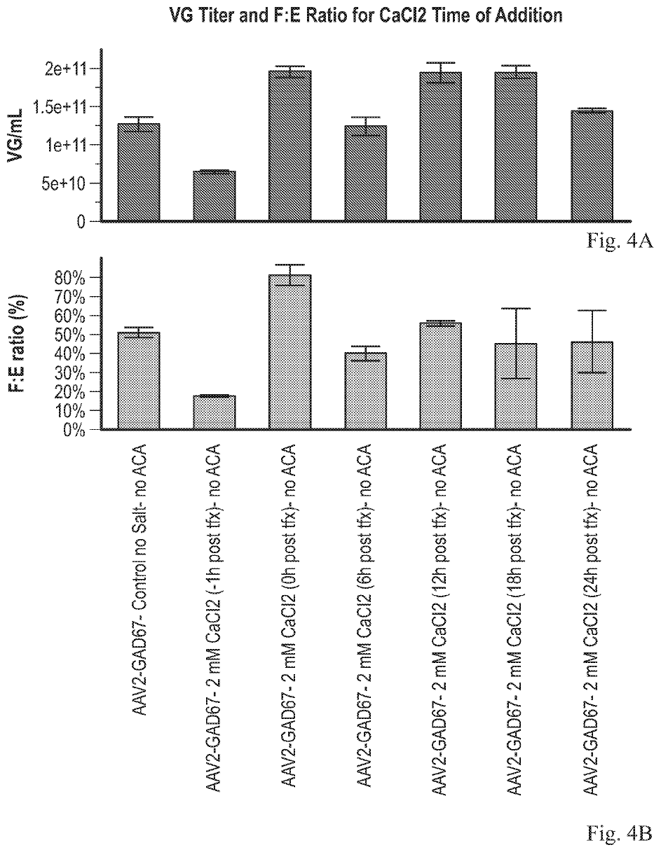

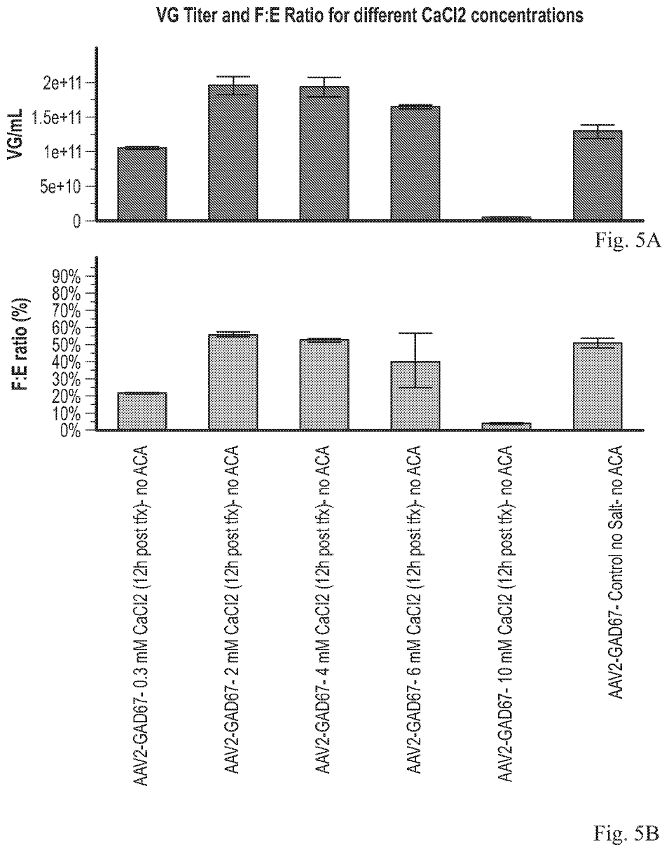

Results: Addition of NaCl or MgCl₂ to the production phase culture medium did not result in increases in VG titer or F:E capsid ratio (FIGS. 3A and 3B). Calcium supplementation one hour prior to transfection by adding CaCl₂) to 2 mM significantly decreased VG titer and F:E capsid ratio compared to control (with no calcium supplementation). See FIGS. 4A and 4B. Calcium supplementation by adding CaCl₂) to 2 mM at 0 to 18 hours post-transfection provided up to a 1.5 fold increase in VG titer compared to control. See FIG. 4A. Increase in F:E capsid ratio was observed when CaCl₂) was added at 0 hours post-transfection. See FIG. 4B. Improvements in VG titre were observed when CaCl₂) was added to a final concentration of 2 to 6 mM (FIG. 5A) and the highest F:E ratio occurred when CaCl₂) was added to a concentration of 2 mM or 4 mM (FIG. 5B).

### Example 4: Production of AAV2, AAV5, and AAV8 in 250 ml Bioreactors

Production of rAAV2-GAD67, rAAV5-RPE65, and rAAV8-CNGB3 was performed in stirred tank bioreactors at 250 mL scale to assess the effect of addition of CaCl₂) on VG titer and F:E ratio. Methods for seed train, expansion, transfection, production and lysis were performed as described in the Example 3 above. Specifically, CaCl₂) was added to production cultures to 2 mM at 12 hours post-transfection. No ACA was added post-transfection to any conditions. Production phase was ended and cells were lysed at 96 hours post-transfection.

### Results:

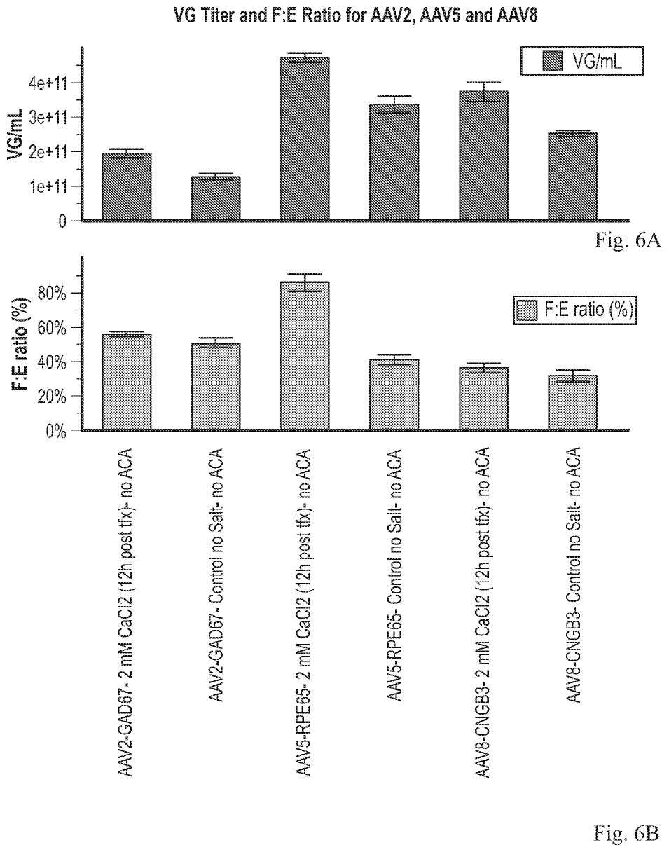

Calcium supplementation by addition of CaCl₂) to 2 mM at 12 hour post-transfection led to improvements in VG titer of 1.5 fold, 1.4 fold, and 1.5 fold for rAAV2, rAAV5, and rAAV8, respectively (FIG. 6A). In addition, calcium supplementation led to a 3-fold increase in F:E ratio for rAAV5 (FIG. 6B).

We claim:

1. A method of producing recombinant adeno-associated virus (rAAV) particles, the method comprising:
(i) an expansion phase comprising increasing the number of cells in at least one culture vessel containing culture medium;
(ii) introducing into the cells a first polynucleotide sequence comprising a transgene flanked by AAV inverted terminal repeats (ITRs), and optionally a second polynucleotide sequence comprising AAV rep and cap genes, and/or a third polynucleotide sequence comprising one or more helper genes;
(iii) a production phase, wherein rAAV particles are produced, comprising culturing the cells from step (ii) and adding calcium (Ca) ions to the culture medium at about 0 to about 24 hours after step (ii) such that the concentration of Ca ions in the culture medium is greater than 0.3 mM and less than 10 mM; and
(iv) isolating the rAAV particles.

2. The method of claim 1, wherein adding Ca ions to the production phase culture medium comprises adding a calcium salt.

3. The method of claim 2, wherein the calcium salt comprises CaCl₂).

4. The method according to any one of claims 1 to 3, wherein the Ca ions are added to a concentration of about 0.5 mM to about 6 mM.

5. The method of claim 4, wherein the Ca ions are added to a concentration of about 2 mM to about 6 mM.

6. The method of claim 4, wherein the Ca ions are added to a concentration of about 2 mM to about 4 mM.

7. The method of claim 4, wherein the Ca ions are added to a concentration of about 1 mM to about 3 mM.

8. The method of claim 4, wherein the Ca ions are added to a concentration of about 2 mM.

9. The method according to any one of claims 1 to 8, wherein the Ca ions are added to the production phase culture medium at about 0 hours to about 24 hours after step (ii).

10. The method of claim 4, wherein the Ca ions are added to the production phase culture medium at about 6 hours to about 18 hours after step (ii).

11. The method according to any one of claims 1 to 3, wherein the Ca ions are added at about 12 hours after step (ii) to a concentration of Ca ions in the culture medium of about 2 mM.

12. The method according to any one of claims 1 to 11, wherein the production phase (step iii) is at least about 48 hours, about 72 to about 100 hours, about 90 hours to about 100 hours, about 92 hours to about 98 hours, about 94 to about 98 hours, or about 96 hours.

13. The method according to any one of claims 1 to 12, wherein the production phase comprises adding one or more of (a) glutamine, a glutamine precursor or an amino acid dipeptide comprising glutamine; and/or (b) sorbitol to the production phase medium.

14. The method of claim 13, wherein the one or more of glutamine, glutamine precursor or an amino acid dipeptide comprising glutamine is selected from one or more of L-alanyl-L-glutamine, L-glutamine, glutamate, glycyl-L-glutamine, glutamine protein hydrolysate, L-glutamic acid, and a glutamine dipeptide.

15. The method of claim 13, wherein at least one of glutamine, glutamine precursor or an amino acid dipeptide comprising glutamine is added to the culture medium at one or more of about 6 hour, about 12 hours, about 24 hours, about 48 hours or about 72 hours after step (ii).

16. The method according to any one of claims 1 to 15, wherein anti-clumping supplement is added to the culture medium in step (i).

17. The method according to any one of claims 1 to 16, wherein at least one of the first, second and third polynucleotide sequences are introduced into the cells by transfection of one or more vectors comprising the first, second and third polynucleotide sequences, by infection with one or more viruses comprising one or more of the first, second and third polynucleotide sequences, by a combination of transfection of the one or more vectors and infection of the one or more viruses, or by electroporation with the first, second and third polynucleotide sequences.

18. The method of claim 17, wherein at least one of the first, second and third polynucleotide sequences are introduced into the cells by transfection.

19. The method according to any one of claims 1 to 18, wherein the transgene encodes a therapeutic protein or a reporter protein.

20. The method of claim 19, wherein the transgene is selected from the group consisting of:
RPE65, RPGR, GAD65, GAD67, and CNGB3.

21. The method according to any one of claims 1 to 20, wherein the AAV ITRs are AAV2 ITRs.

22. The method according to any one of claims 1 to 21, wherein the AAV cap gene is from an AAV serotype or AAV variant selected from the group consisting of: AAV1, AAV2, AAV3, AAV4, AAV5, AAV6, AAV7, AAV8, AAV9, AAV10, AAV 11, AAV12, AAV13, AAVrh10, AAV-PHP.5, AAV-PHP.B, AAV-PHP.eB, AAV2-retro, AAV9-retro, and a hybrid thereof.

23. The method according to any one of claims 1 to 22, wherein the cells are mammalian cells.

24. The method of claim 23, wherein the mammalian cells are HEK293 cells.

25. The method of claim 23 or 24, wherein the cells are cultured in suspension.

26. The method of claim 25, wherein the at least one culture vessel is a shaker flask, a spinner flask, a cellbag or a bioreactor.

27. The method according to any one of the preceding claims, wherein in step (i) the culture medium has a pH of about 7.2 to about 7.4 and culturing the cells comprises CO₂ sparging.

28. The method of claim 27, wherein prior to step (ii) the pH of the culture medium is changed to about 6.9 and CO₂ sparging is halted.

29. The method according to any one of the preceding claims, wherein the step of isolating the rAAV particles comprises lysing the cells and optionally clarifying the resulting lysate and subjecting the clarified lysate to a purification step.

30. The method of claim 29, wherein the purification step is selected from the group consisting of: ultracentrifugation, affinity chromatography, and ion-exchange chromatography.

31. A population of rAAV particles produced by a method according to any one of claims 1 to 30.

32. A pharmaceutical composition comprising the population of rAAV particles of claim 31.
